(19) United States
(12) Patent Application Publication
(10) Pub. No.: US 2025/0339127 A1
(43) Pub. Date: Nov. 6, 2025

(54) INTRALUMINAL IMAGING DEVICES WITH ELECTROMAGNETIC POSITION TRACKING AND REDUCED ELECTROMAGNETIC NOISE INTERFERENCE

(71) Applicant: KONINKLIJKE PHILIPS N.V., EINDHOVEN (NL)

(72) Inventor: Nicholas Andrew STROM, MAPLE GROVE, MN (US)

(21) Appl. No.: 19/196,215

(22) Filed: May 1, 2025

Related U.S. Application Data
(60) Provisional application No. 63/641,441, filed on May 2, 2024.

(51) Int. Cl. A61B 8/00; A61B 8/08; A61B 8/12; A61M 25/00; A61M 25/01

(52) U.S. Cl. CPC A61B 8/445; A61B 8/0883; A61B 8/12; A61B 8/4254; A61M 25/005; A61M 25/0127

(57) ABSTRACT

Intraluminal imaging devices configured for electromagnetic position tracking are provided. In some aspects, intracardiac echocardiography (ICE) catheters, intravascular ultrasound (IVUS) catheters, and/or other imaging devices having electromagnetic position tracking capabilities with reduced electromagnetic noise interference are provided. In some aspects, an intraluminal imaging device includes at least one of a shield having a magnetic permeability between about 80,000 and about 400,000 extending along a length of the device or a multiplexer positioned within a handle or the proximal portion of the device and configured to remove electromagnetically induced noise.

### TECHNICAL FIELD

The present disclosure relates generally to intraluminal imaging devices configured for electromagnetic position tracking. In some aspects, intracardiac echocardiography (ICE) catheters, intravascular ultrasound (IVUS) catheters, and/or other imaging devices having electromagnetic position tracking capabilities with reduced electromagnetic noise interference are provided.

### BACKGROUND

Diagnostic and therapeutic ultrasound catheters have been designed for use inside many areas of the human body. In the cardiovascular system, two common diagnostic ultrasound methods are intravascular ultrasound (IVUS) and intra-cardiac echocardiography (ICE). In some implementations, a single rotating transducer or an array of transducer elements is used to transmit ultrasound at the distal portion of the imaging catheters. The same transducers and/or separate transducers may be used to receive echoes from the tissue. A signal generated from the echoes is transferred to a console which allows for the processing, storing, displaying, and/or manipulating the ultrasound-related data.

IVUS catheters are often used in the large and small blood vessels (arteries or veins) of the body and are almost always delivered over a guidewire having a flexible tip. ICE catheters are often used to image chambers of the heart and surrounding structures, for example, to guide and facilitate medical procedures, such as transseptal lumen punctures, left atrial appendage closures, atrial fibrillation ablation, and valve repairs. Commercially available ICE catheters are typically not designed to be delivered over a guidewire, but instead have distal ends that can be articulated by a steering mechanism located in a handle at the proximal end of the catheter. For example, an ICE catheter may be inserted through the femoral or jugular artery when accessing the anatomy and steered in the heart to acquire images beneficial for ensuring the safety of the associated medical procedures.

ICE catheters, like many other intraluminal imaging catheters, are typically controlled by an operator at the operating table on which the patient is positioned. The ICE catheter may be inserted into a lumen of the patient, such as a blood vessel, and an imaging tip of the catheter may be navigated through the vasculature to a desired location to image a region of interest. The ICE catheter may be navigated by maneuvering a handle attached to the ICE catheter and/or by manipulating one or more movement controls disposed on the handle. This process can take time, as the operator (e.g., physician) must orient themselves in complex anatomy and make medical decisions surrounding diagnosis and/or treatment. In some instances, the position of an imaging face or transducer array of the ICE catheter may be changed by small rotational and/or translational movement of the ICE catheter handle. Therefore, it can be desirable to know the location and/or orientation of the imaging tip of the ICE catheter during a procedure.

### SUMMARY

The invention provides devices, systems, and related methods that overcome the limitations associated with existing designs and provide intraluminal imaging devices having electromagnetic position tracking capabilities with reduced electromagnetic noise interference. In some aspects, the intraluminal imaging device includes at least one electromagnetic noise reduction mechanism configured to reduce electromagnetically induced noise in wires extending from each of one or more electromagnetic position sensors. The electromagnetic noise reduction mechanism may include a shield configured to prevent electromagnetically induced noise in the wires. The shield may be formed of a material having a magnetic permeability between about 80,000 and about 400,000, or other suitable value. The electromagnetic noise reduction mechanism may include a multiplexer in addition to or in lieu of the shield. The multiplexer may be positioned in a proximal portion or handle of the intraluminal imaging device and be configured to process signals carried by the wires extending from each of the one or more electromagnetic position sensors to remove electromagnetically induced noise.

In some aspects, an intraluminal imaging device is provided. The intraluminal imaging device may comprise a flexible elongate member; an imaging core coupled to a distal portion of the flexible elongate member; one or more electromagnetic position sensors coupled to the distal portion of the flexible elongate member, the one or more electromagnetic position sensors having a known position relative to at least one of the imaging core, the distal portion of the flexible elongate member, or another electromagnetic position sensor of the one or more electromagnetic position sensors; a handle coupled to a proximal portion of the flexible elongate member; a pair of wires extending from each of the one or more electromagnetic position sensors to a proximal portion of the flexible elongate member; and a shield extending along a length of the flexible elongate member from the distal portion to the proximal portion, the shield having a magnetic permeability between about 80,000 and about 400,000.

The shield may include a cylindrical braid and/or a coil. The shield may comprise at least one of a mu-metal or a metallic glass alloy. The shield may surround the pair of wires extending from each of the one or more electromagnetic position sensors to the proximal portion of the flexible elongate member. In some aspects, the shield may be embedded within a tubular wall of the flexible elongate member. In this regard, the tubular wall may define an outer surface of the flexible elongate member. In some aspects, the shield may surround a plurality of wires extending from the imaging core to the proximal portion of the flexible elongate member. In some aspects, a plurality of wires extending from the imaging core to the proximal portion of the flexible elongate member are positioned outside of the shield. In some aspects, the intraluminal imaging device includes a multiplexer positioned within the handle or the proximal portion of the flexible elongate member, the multiplexer configured to process signals carried by the pair of wires extending from each of the one or more electromagnetic position sensors to remove electromagnetically induced noise.

In some aspects, the intraluminal imaging device may comprise a flexible elongate member; an imaging core coupled to a distal portion of the flexible elongate member; one or more electromagnetic position sensors coupled to the distal portion of the flexible elongate member, the one or more electromagnetic position sensors having a known position relative to at least one of the imaging core, the distal portion of the flexible elongate member, or another electromagnetic position sensor of the one or more electromagnetic position sensors; a handle coupled to a proximal portion of the flexible elongate member; a pair of wires extending from each of the one or more electromagnetic position sensors to a proximal portion of the flexible elongate member; and a multiplexer positioned within the handle or the proximal portion of the flexible elongate member, the multiplexer configured to process signals carried by the pair of wires extending from each of the one or more electromagnetic position sensors to remove electromagnetically induced noise.

The multiplexer may be configured to perform time-division multiplexing with a time slot length between about 0.25 microseconds and about 1.0 microsecond. The multiplexer may be configured to perform the time-division multiplexing by subtracting a signal based on an immediately preceding time slot from a signal based on a current time slot. The multiplexer may be further configured to receive analog input signals and output analog output signals, wherein the analog input signals include the signal based on the immediately preceding time slot and the signal based on the current time slot.

In some aspects, an apparatus is provided. The apparatus may comprise an intracardiac catheter sized and shaped for advancement through a blood vessel; an imaging core coupled to a distal portion of the intracardiac catheter; one or more electromagnetic position sensors coupled to the distal portion of the intracardiac catheter, the one or more electromagnetic position sensors having a known position relative to at least one of the imaging core, the distal portion of the intracardiac catheter, or another electromagnetic position sensor of the one or more electromagnetic position sensors; a handle coupled to a proximal portion of the intracardiac catheter; a pair of wires extending from each of the one or more electromagnetic position sensors to a proximal portion of the intracardiac catheter; a shield extending along a length of the intracardiac catheter from the distal portion to the proximal portion, the shield having a magnetic permeability between about 80,000 and about 400,000; and a multiplexer positioned within the handle or the proximal portion of the intracardiac catheter, the multiplexer configured to process signals carried by the pair of wires extending from each of the one or more electromagnetic position sensors to remove electromagnetically induced noise.

Additional aspects, features, and advantages of the present disclosure will become apparent from the following detailed description.

### BRIEF DESCRIPTION OF THE DRAWINGS

Illustrative aspects of the present disclosure will be described with reference to the accompanying drawings, of which:

FIG. 1 a schematic diagram of an intra-cardiac echocardiography (ICE) imaging system according to aspects of the present disclosure.

FIG. 2 is a schematic diagram of a portion of an ICE device according to aspects of the present disclosure.

FIG. 3 is a schematic diagram illustrating deflections of an ICE device according to aspects of the present disclosure.

FIG. 4 is a top view showing an intraluminal imaging device with electromagnetic position tracking capabilities being used during an intraluminal imaging procedure according to aspects of the present disclosure.

FIG. 5 is a cross-sectional end view of a body of an intraluminal imaging device according to aspects of the present disclosure.

FIG. 6 is a cross-sectional longitudinal side view of a body of an intraluminal imaging device according to aspects of the present disclosure.

FIG. 7 is a cross-sectional end view of a body of an intraluminal imaging device according to aspects of the present disclosure.

FIG. 8 is a cross-sectional end view of a body of an intraluminal imaging device according to aspects of the present disclosure.

FIG. 9 is a cross-sectional end view of a body of an intraluminal imaging device according to aspects of the present disclosure.

FIG. 10 is a schematic diagram of an intraluminal imaging system according to aspects of the present disclosure.

### DETAILED DESCRIPTION

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the ICE system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other aspects of the present disclosure. For the sake of brevity, however, the numerous iterations of these combinations will not be described separately.

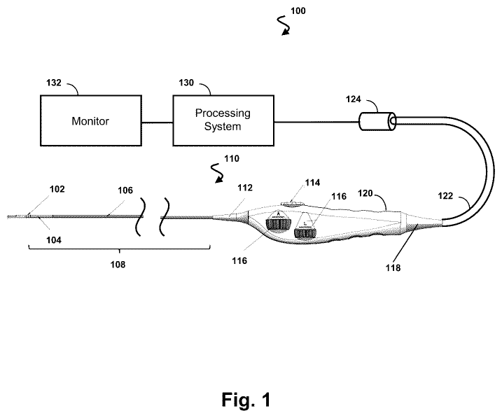

FIG. 1 is a schematic diagram of an intra-cardiac echocardiography (ICE) imaging system 100 according to aspects of the present disclosure. The system 100 may include an ICE device 110 (e.g., an ICE catheter and/or other intraluminal imaging device), a connector 124, a control and processing system 130, such as a console and/or a computer, and a monitor 132. The ICE device 110 may include a tip assembly 102, a flexible elongate member 108, and a handle 120. The flexible elongate member 108 may include a distal portion 104 and a proximal portion 106. The distal end of the distal portion 104 may be attached to the tip assembly 102. The proximal end of the proximal portion 106 may be attached to the handle 120. For example, in some instances a resilient strain relief 112 couples the proximal portion 106 to the handle 120. The handle 120 may be used for manipulation of the ICE device 110 and/or manual control of the ICE device 110. The handle 120 can include actuators 116, a clutch 114, and other steering control components for steering the ICE device 110, such as deflecting the tip assembly 102 and the distal portion 104. In some aspects, the ICE device 110 may include steering and/or control mechanisms similar to those described in U.S. Pat. No. 11,464,481, which is hereby incorporated by reference in its entirety. The tip assembly 102 may include an imaging array, imaging core, and/or imaging sensor with a plurality of ultrasound transducer elements and associated circuitry.

The handle 120 may be connected to the connector 124 via a strain relief 118 and an electrical cable 122. The connector 124 may be configured in any suitable configurations (including wired and/or wireless communications) to interconnect with the processing system 130 and the monitor 132 for processing, storing, analyzing, manipulating, and displaying data obtained from signals generated by the imaging core at the tip assembly 102. The processing system 130 can include one or more processors, memory, one or more input devices, such as keyboards and any suitable command control interface device. The processing system 130 can be operable to facilitate the features of the system 100 described herein. For example, the processor can execute computer readable instructions stored on the non-transitory tangible computer readable medium. The monitor 132 can be any suitable display device, such as liquid-crystal display (LCD) panel or the like.

In operation, a physician or a clinician advances the flexible elongate member 108 into a vessel within a heart anatomy. The tip assembly 102 and the flexible elongate member 108 may be shaped and sized for insertion into vessels of a patient body. The flexible elongate member 108 may be composed of any suitable material, such as Pebax® polyether block amides. The distal portion 104 and the proximal portion 106 may be tubular in shape and may include a primary lumen and one or more pullwire lumens extending longitudinally along the flexible elongate member 108. The primary lumen may be sized and shaped to accommodate an electrical cable interconnecting the tip assembly 102 and the connector 124 for transferring data (e.g., echo signals) obtained from the transducer elements. In some aspects, the primary lumen can be sized and shaped to accommodate other components for diagnostic and/or therapy procedures. The pullwire lumens may be sized and shaped to accommodate pullwires, for example, extending from the distal portion 104 to the handle 120. The pullwires may be coupled to the actuators 116 and/or the clutch 114 such that the flexible elongate member 108 and the tip assembly 102 are deflectable based on actuations of the actuators 116 and/or the clutch 114. In some instances, the primary lumen may be sized and shaped to facilitate alignment of the pullwire lumens. In addition, the tubular body of the flexible elongate member 108 may include a lined variable braided reinforcement layer configured to provide flexibility and kink resistance.

Dimensions of the flexible elongate member 108 can vary in different aspects. Generally, the flexible elongate member 108 may be positioned within any lumen or area within a patient body. In some instances, the flexible elongate member 108 may be sized and/or shaped for positioning with one or more particular lumens and/or target areas with the patient body. In some aspects, the flexible elongate member 108 can be a catheter having an outer diameter between about 8 and about 12 French (Fr) and can have a total length between about 80 centimeters (cm) to about 120 cm, where the proximal portion 106 can have a length between about 70 cm to about 118 cm and the distal portion 104 can have a length between about 2 cm to about 10 cm. While aspects described herein may refer to the ICE device 110, the concepts of the present disclosure may be applied to other types of intraluminal imaging devices, including IVUS, OCT, and/or other imaging modalities.

The physician or clinician can steer the flexible elongate member 108 to a desired position within the patient body. In this regard, the desired position may be near an area of interest to be imaged by the ICE device 110. In some aspects, the physician or clinician steers the flexible elongate member 108 by controlling the actuators 116 and the clutch 114 on the handle 120. For example, one actuator 116 may deflect the tip assembly 102 and the distal portion 104 in a left-right plane and the other actuator 116 may deflect the tip assembly 102 and the distal portion 104 in an anterior-posterior plane. The clutch 114 may provide a locking mechanism to lock the positions of the actuators 116 and, in turn, the deflection of the flexible elongate member 108 while imaging the area of interest.

The imaging process may include activating the ultrasound transducer elements of the tip assembly 102 to produce ultrasonic energy. A portion of the ultrasonic energy may be reflected by the area of interest and the surrounding anatomy. The ultrasound echo signals may be received by the ultrasound transducer elements. The connector 124 may transfer the received echo signals to the processing system 130. The processing system 130 may process the received echo signals to generate the ultrasound image(s) and output the image(s) to the monitor 132 for display. In some aspects, the processing system 130 may control the activation of the ultrasound transducer elements and/or the repletion of the echo signals. In some aspects, the processing system 130 and the monitor 132 may be part of the same system.

The system 100 may be utilized in a variety of applications such as transseptal lumen punctures, left atrial appendage closures, atrial fibrillation ablation, and valve repairs and can be used to image vessels and structures within a patient body. Although the system 100 is described in the context of ICE catheterization procedures, the system 100 is suitable for use with any catheterization procedure, including structural heart, cardiac, peripheral, and/or otherwise. In addition, the tip assembly 102 may include any suitable physiological sensor, component, and/or functional element for diagnosis, treatment, and/or therapy, such as pressure sensor(s), flow sensor(s), force sensor(s), doppler sensor(s), etc. The physiological sensors may be provided in addition to and/or in lieu of the imaging element(s). Thus, the handle 120 can be used to guide articulation and/or positioning of any type of functional element included in the distal portion 104 of the ICE device 110.

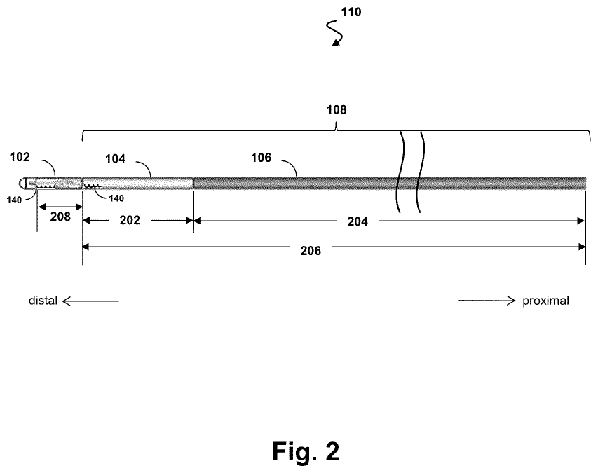

FIG. 2 is a schematic diagram of a portion of the ICE device 110 according to aspects of the present disclosure. The tip assembly 102 and the flexible elongate member 108 are shaped and sized for insertion into vessels of a patient body. The flexible elongate member 108 can be composed of any suitable material, such as polyether block amides. Polyether block amides are commonly manufactured under the tradename Pebax®. The distal portion 104 and the proximal portion 106 may be tubular in shape and may include a primary lumen and one or more pullwire lumens extending longitudinally along the flexible elongate member 108.

In some aspects, the tip assembly 102 and/or the distal portion 104 of the flexible elongate member 108 includes one or more electromagnetic position sensors 140. The electromagnetic position sensors 140 may be a five degree of freedom (5 DOF) sensor. In some aspects, the electromagnetic position sensors 140 may be an electrical inductor (e.g., coil or other suitable structure). The electromagnetic position sensors 140 may be placed in a known mechanical position within the ICE device 110. More than one electromagnetic position sensor 140 can be utilized to gain a sixth degree of freedom (6 DOF). In this regard, all relevant guidance positions and orientations may be captured with 6 DOF, including X, Y, Z, roll, pitch, and yaw. Aspects of the present disclosure may be particularly suitable for smaller electromagnetic positions sensors 140 that may be more susceptible to electromagnetic noise interference. For example, in some instances the electromagnetic positions sensors 140 may have a length between about 1.5 mm and about 20 mm and a diameter between about 0.2 mm and about 0.8 mm, or other suitable dimensions. In some particular applications in accordance with the present disclosure, the electromagnetic position sensors 140 may include a coil having a length of approximately 2.5 mm and a diameter of approximately 0.3 mm, though any other suitable combinations of length and diameter (or width and height) may be utilized. Further, in some instances the electromagnetic positions sensors 140 may be made of a material (or combination of materials) suitable for measuring characteristics of an electromagnetic field (e.g., magnetic field flux, magnetic field differential etc.).

The electromagnetic position sensors 140 may be in a known, fixed position relative to a target of the ICE device 110 that is to be tracked. For example, the electromagnetic position sensors 140 may have a known position relative to the imaging core, the distal portion of the flexible elongate member, another electromagnetic position sensor, the distal most tip, a boundary and/or middle of the tip assembly, a boundary and/or middle of the imaging core, a radiopaque marker, and/or other aspects or components of the ICE device 110. For example, as shown in FIG. 2, the electromagnetic position sensor 140 within the tip assembly 102 is spaced from the electromagnetic position sensor 140 within the distal portion 104 of the flexible elongate member 108 by a fixed distance 208. Similarly, the electromagnetic position sensor 140 within the tip assembly 102 may be spaced from the imaging core (e.g., proximal end, distal end, or middle) and/or a distal most tip of the ICE device 110 by a known distance. Further, the relative positional orientations of the electromagnetic positions sensors 140 may be known. For example, in some aspects the electromagnetic position sensor 140 within the tip assembly 102 may extend substantially colinear with or parallel to the electromagnetic position sensor 140 within the distal portion 104 of the flexible elongate member 108. In other aspects, the electromagnetic position sensor 140 within the tip assembly 102 may extend substantially perpendicular to the electromagnetic position sensor 140 within the distal portion 104 of the flexible elongate member 108. In yet other aspects, the electromagnetic position sensor 140 within the tip assembly 102 may extend at an oblique angle relative to the electromagnetic position sensor 140 within the distal portion 104 of the flexible elongate member 108. Similar orientation approaches may be used for two or more electromagnetic position sensors 140 within the tip assembly 102 and/or within the distal portion 104 of the flexible elongate member 108. In this regard, the known, but different orientations of the electromagnetic position sensors 140 may be utilized to determine the location and/or orientation of the tip assembly 102 and/or an associated imaging core during a medical procedure utilizing magnetic fields produced by an electromagnetic field generator of a position tracking system.

The primary lumen of the ICE device 110 may be sized and shaped to accommodate a pair of wires extending from each of the electromagnetic position sensors 140 to the proximal portion 106 of the flexible elongate member 108. The primary lumen may be further sized and shaped to accommodate a plurality of wires (e.g., an electrical cable) interconnecting an imaging core of the tip assembly 102 to the handle 120 and/or the connector 124 for transferring echo signals obtained from the transducer elements of the imaging core to the processing system 130. In some aspects, the primary lumen can be shaped and sized to accommodate other components for diagnostic and/or therapy procedures.

The pullwire lumens may be sized and shaped to accommodate pullwires, for example, extending from the distal portion 104 to the handle 120. The pullwires may be coupled to the actuators 116 and the clutch 114 such that the flexible elongate member 108 and the tip assembly 102 are deflectable based on actuations of the actuators 116 and the clutch 114. In an embodiment, the primary lumen is shaped to facilitate alignment of the pullwire lumens. In addition, the tubular body of the flexible elongate member 108 may include a lined variable braided reinforcement layer configured to provide flexibility and kink resistance. The arrangements and configurations of the pullwires, the primary lumen, the pullwire lumens, the tip assembly 102, and the lined variable braided reinforcement layer are described in greater details herein. Dimensions of the flexible elongate member 108 can vary in different aspects. In some aspects, the flexible elongate member 108 can be a catheter having an outer diameter between about 8 and about 12 French (Fr) and can have a total length 206 between about 80 centimeters (cm) to about 120 cm, where the proximal portion 106 can have a length 204 between about 70 cm to about 118 cm and the distal portion 104 can have a length 202 between about 2 cm to about 10 cm.

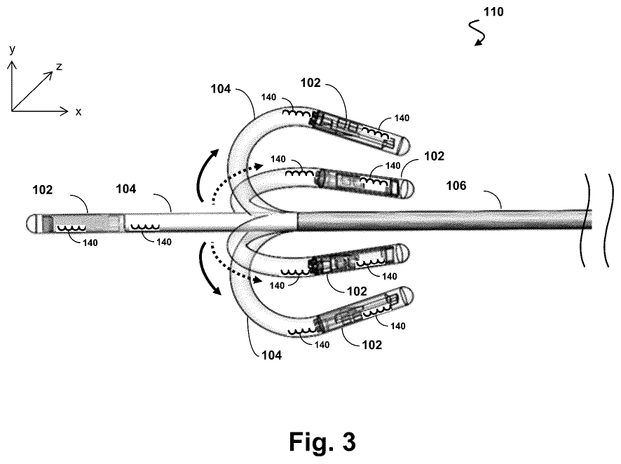

FIG. 3 is a schematic diagram illustrating deflections planes of the ICE device 110 according to aspects of the present disclosure. In some aspects, the orientation of the flexible elongate member 108 shown in FIG. 2 may be referred to as a neutral position. In FIG. 3, the tip assembly 102 and the distal portion 104 of the flexible elongate member 108 may be deflected from the neutral position to one or more other positions. As shown, the tip assembly 102 and the distal portion 104 can be deflected along a first plane as shown by the solid arrows and a second plane as shown by the dotted arrows. In FIG. 3, the first plane is represented by an x-y plane and the second plane is represented by an x-z plane. For example, the x-y plane may correspond to a left-right plane and the x-z plane may correspond to an anterior-posterior plane for imaging the heart anatomy.

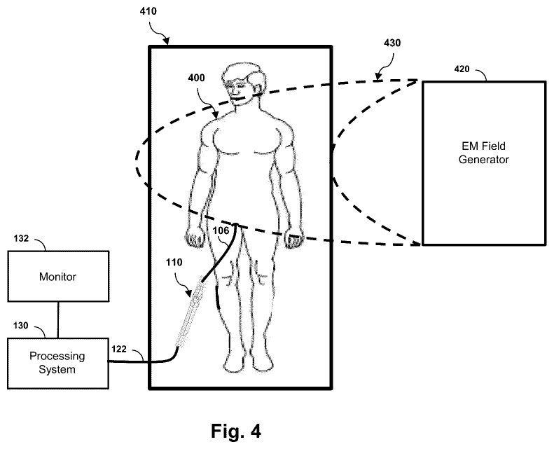

FIG. 4 is a top view showing an intraluminal imaging device with electromagnetic position tracking capabilities being used during an intraluminal imaging procedure according to aspects of the present disclosure. In this regard, the intraluminal imaging device (e.g., ICE catheter, IVUS catheter, OCT device, and/or other imaging device) may be inserted into a body of a patient 400. For example, a distal portion of the intraluminal imaging device can be positioned within any suitable lumen with the body of a patient. In some instances, the distal portion of the intraluminal imaging device is advanced through the femoral or jugular artery when accessing the anatomy of the patient 400 and steered to the heart to acquire images for associated medical procedures. For example, as shown in FIG. 4, a distal portion of an ICE device 110 having an imaging core or assembly may be inserted into a femoral artery of the patient 400 on an operating table 410 and advanced to a desired location within the body. In some instances, at least a section of the proximal portion 106 of the ICE device 110 may remain outside of the patient 400, as shown.

Advancing the distal portion to the intraluminal imaging device to the desired location within the patient 400 may include steering the distal portion of the intraluminal imaging device using one or more steering mechanisms of the handle. One or more components of a steering mechanism can include pulley(s) coupled to the pullwire segment(s), axle(s), and/or actuation control member(s). The actuation control members can be coupled to the pullwire segments via the pulleys such that movement of the actuation control members causes corresponding deflection of the distal portion of the flexible elongate member. A clutch mechanism may include a clutch control member, a clutch cam, a clutch spring, and frictional members. The clutch control member can be moved to increase or decrease the compression force on the clutch cam. The clutch cam in turn applies the suitable compression force on the clutch spring. The frictional members may be positioned adjacent to and/or in contact with the actuation control members. The actuation control members may be urged into contact with the frictional members in response to the control force. Increased contact slows down the rate of return to the non-deflected state. Decreased contact speeds up the rate of return to the non-deflected state. In this manner, a user may control the rate at which the distal portion of the intraluminal imaging device returns to a non-deflected state using the clutch mechanism.

Advancing the distal portion to the intraluminal imaging device to the desired location within the patient 400 may also include tracking the position and/or orientation of the distal portion and/or tip assembly of the intraluminal imaging device. In this regard, the intraluminal imaging device may include one or more electromagnetic position sensors as described previously. An electromagnetic field generator 420 may generate electromagnetic fields 430 that are used to determine the position and/or orientation of the electromagnetic position sensors and, thereby, the associated position and/or orientation of one or more aspects of the intraluminal imaging device. In this regard, the position and/or orientation of the electromagnetic position sensors may be determined by measuring an induced current produced in the electromagnetic position sensors by the electromagnetic fields 430. In this regard, a pair of wires may carry a signal corresponding to the induced current from the electromagnetic position sensors to a proximal portion and/or the handle of the intraluminal imaging device.

In use, the pair of wires extending from the electromagnetic position sensors may act as antennae and pick up undesired signals or noise (e.g., due to electromagnetic interference, the electromagnetic fields 430 generated by the electromagnetic field generator 420, or otherwise). A method for attempting to eliminate noise in this context is to twist the pair of wires around one another. In this regard, twisted pair cables may include two insulated copper wires twisted around each other. In theory, any external electromagnetically induced noise should be carried equally on both wires such that the receiving system can subtract one signal from the other to cancel out the noise. However, in practice, relying on a twisted pair of wires to eliminate noise has proven insufficient in the context of electromagnetic position sensors within intraluminal imaging devices. This is especially true for smaller electromagnetic position sensors (e.g., sensors having a length of between about 2.0-3.0 mm and a diameter of between about 0.25-0.35 mm) that may have relatively weaker signal-to-noise ratios and, therefore, may be more susceptible to interference causing the associated signals reducing the spatial accuracy of the determined position and/or orientation of the electromagnetic position sensors within the electromagnetic fields 430. There is a desire to minimize the size of intraluminal imaging devices for many reasons. As the size is minimized, certain components (such as the electromagnetic position sensors) must also be minimized to achieve the desired size goals. However, generally the smaller the electromagnetic position sensor, the smaller the signal acquired/generated by the electromagnetic position sensor in response to an electromagnetic field, which can lead to a lower signal-to-noise ratio. Aspects of the present disclosure may be particularly suitable for these smaller electromagnetic positions sensors that may be more susceptible to electromagnetic noise interference.

A twisted pair of conductor wires may have imperfections. During spooling/unspooling and handling of the twisted pair it is possible for portions of the wires to become untwisted. This portion of untwisted wire can act as an antenna and pick up undesired electromagnetic interference and/or current from an induced magnetic field (e.g., electromagnetic fields 430 generated by the electromagnetic field generator 420 and/or electromagnetic fields generated by other electromagnetic components in (or adjacent to) the procedure room). Further, off-the-shelf twisted pair cables are not optimized for use in an intraluminal imaging device with electromagnetic position tracking capabilities. For example, many twisted pair cables are unshielded, while others use shielding materials such as an aluminum (foil) wrap or a copper braid that have a relative magnetic permeability of approximately 1.0. As a result, the ability for even shielded twisted pair cables to prevent interference from external magnetic fields may be very low.

In some aspects, the present disclosure provides intracardiac echocardiography (ICE) catheters, intravascular ultrasound (IVUS) catheters, and/or other imaging devices having electromagnetic position tracking capabilities with reduced electromagnetic noise interference. For example, an intraluminal imaging device may comprise a flexible elongate member, an imaging core coupled to a distal portion of the flexible elongate member, one or more electromagnetic position sensors coupled to the distal portion of the flexible elongate member, a pair of wires extending from each of the one or more electromagnetic position sensors to a proximal portion of the flexible elongate member; and at least one electromagnetic noise reduction mechanism configured to reduce electromagnetically induced noise in the pair of wires extending from each of the one or more electromagnetic position sensors. In some instances, the electromagnetic noise reduction mechanism includes at least one of a shield having a magnetic permeability between about 80,000 and about 400,000, including around 100,000 or other suitable value, and/or a multiplexer configured to process signals carried by the pair of wires extending from each of the one or more electromagnetic position sensors to remove electromagnetically induced noise.

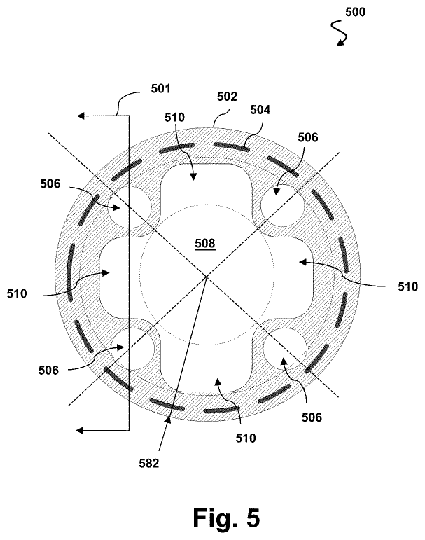

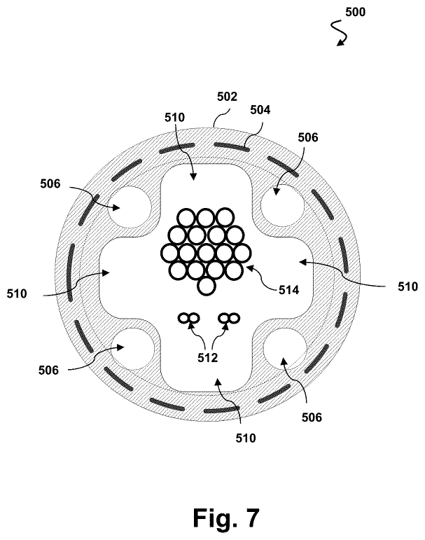

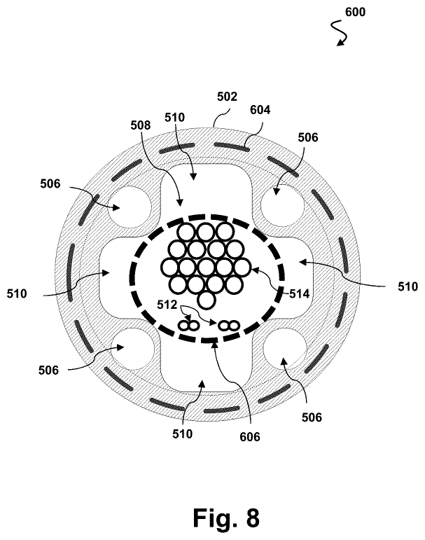

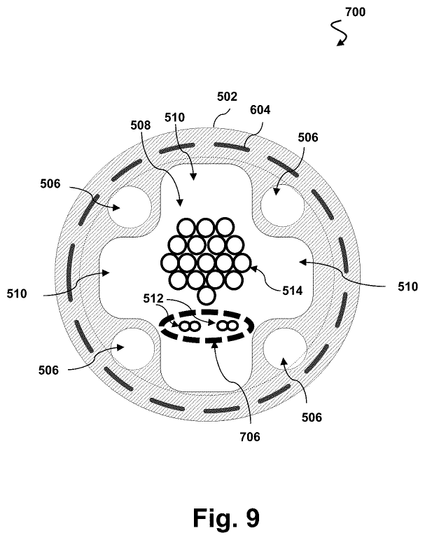

In some aspects, the electromagnetic noise reduction mechanism includes the shield. The shield may extend along a length of the flexible elongate member from the distal portion to the proximal portion. The shield may comprise a cylindrical braid, a coil, a weave, and/or other suitable configuration. In this regard, the shield may partially and/or fully surround the wires extending from the one or more electromagnetic position sensors along a length of the flexible elongate member. In some aspects, the shield may be embedded within a tubular wall of the flexible elongate member (see, e.g., shield 504 of FIGS. 5-7). In this regard, the tubular wall may define an outer surface of the flexible elongate member in some instances. In other instances, the tubular wall may define an inner or intermediate surface of the flexible elongate member. In some aspects, the shield may be positioned within an inner lumen of the flexible elongate member (see, e.g., shields 606 and 706 of FIGS. 8-9, respectively). In some aspects, the shield surrounds a plurality of wires extending from the imaging core to the proximal portion of the flexible elongate member (see, e.g., shields 504 and 606 of FIGS. 7 and 8, respectively). In some aspects, the shield comprises at least one of a mu-metal, a metallic glass alloy, and/or other material having a magnetic permeability between 80,000 and 400,000 or greater.

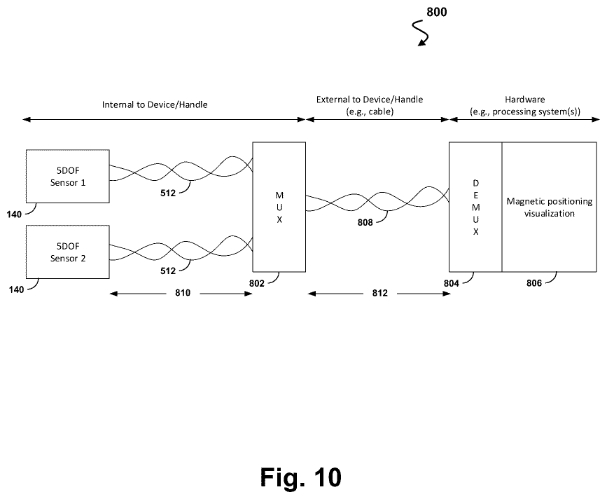

In some aspects, the electromagnetic noise reduction mechanism includes the multiplexer. The multiplexer may be positioned within the proximal portion of the flexible elongate member and/or a handle coupled to the proximal portion of the flexible elongate member (see, e.g., multiplexer 802 of FIG. 10). The multiplexer may configured to perform time-division multiplexing to remove electromagnetic interference. In some aspects, the multiplexer is configured to perform time-division multiplexing with a time slot length between about 0.25 microseconds and about 1.0 microsecond. The multiplexer may be configured to perform the time-division multiplexing by subtracting a signal based on an immediately preceding time slot from a signal based on a current time slot. The multiplexer may be further configured to receive and process analog input signals and output analog output signals. In this regard, the analog input signals include the signals based on the immediately preceding time slot and the signals based on the current time slot utilized in the time-division multiplexing operations. In some instances, the multiplexer and/or a microcontroller in communication with the multiplexer (or implementing the multiplexer functionality) within the handle of the intraluminal imaging device may digitize the resulting signal and utilize one or more outputs (e.g., general purpose outputs of the microcontroller) to interface with the associated electromagnetic position tracking system. In this regard, the signal between the multiplexer and/or microcontroller in the handle of the intraluminal imaging device and the associated electromagnetic position tracking system may be subject to insignificant electromagnetic interference. In this regard, electromagnetic interference external to the intraluminal imaging device (e.g., in a cable extending between the handle of the intraluminal imaging device and a processing system) may be reduced and/or eliminated (e.g., by subtracting time-shifted signals to reduce common noise across multiple wires of the cable). In some aspects, the electromagnetic noise reduction mechanism includes both the multiplexer and the shield.

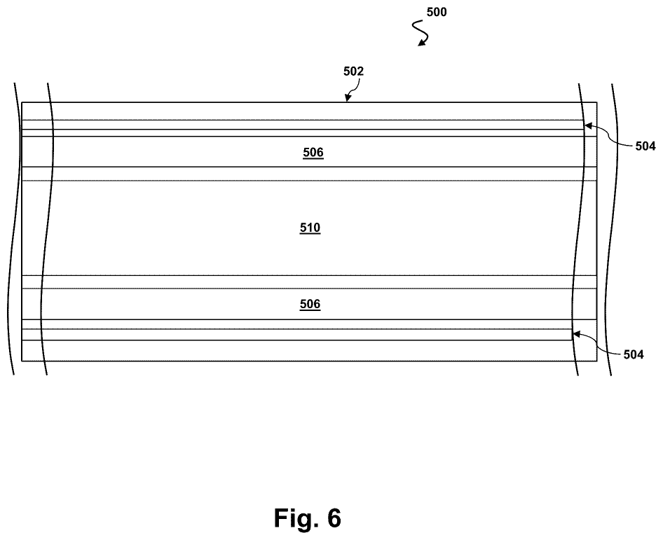

FIGS. 5-7 illustrate aspects of a body 500 (e.g., a multi-lumen catheter shaft) of an intraluminal imaging device according to aspects of the present disclosure. FIG. 5 is a cross-sectional end view of the body 500 according to aspects of the present disclosure, while FIG. 6 is a cross-sectional longitudinal side view of the body according to aspects of the present disclosure. FIG. 7 is a cross-sectional end view of the body 500 with additional components of the intraluminal imaging device shown according to aspects of the present disclosure. In some aspects, the body 500 may be utilized in intracardiac echocardiography (ICE) catheters, intravascular ultrasound (IVUS) catheters, and/or other imaging devices having electromagnetic position tracking capabilities. For example, the body 500 may form part or all of flexible elongate member 108 of the ICE device 110, including the distal portion 104 and/or the proximal portion 106, or sections thereof.

The body 500 may be tubular in shape with a tubular wall 502 and a primary lumen 508. The primary lumen 508 may extend between a proximal end and a distal end, for example, along a central longitudinal axis of the body 500. As shown, the primary lumen 508 may have a cross-shaped cross-sectional profile. In this regard, the primary lumen 508 may include arms 510 extending from a central lumen. The cross-shaped profile may be rounded (as shown in FIG. 5) or rectangular, for example. In some aspects, the primary lumen 508 may have other cross-sectional profiles, including geometrical, non-geometrical, and/or combinations thereof. Further, the cross-section profile of the primary lumen 508 may change along the length of the body 500 in some instances. The dimensions of the primary lumen 508 can be sized to allow components (e.g., a printed circuit board (PCB), a coaxial cable, a plurality of wires, etc.) to be introduced through the primary lumen 508 during assembly, and thus may improve handling responsiveness during operation.

The body 500, including tubular wall 502, may be formed of any suitable material. In some aspects, the tubular wall 502 may be composed of a high durometer polymeric material at a distal segment and a low durometer polymeric material at a proximal segment. For example, the high durometer polymeric material may have a durometer between 63D-80D and include materials such as Pebax® 72D or a suitable nylon. The low durometer polymeric material may have a durometer between 30D to 55D and include materials such as Pebax® 35D, Pebax® 45D, or a suitable nylon. The differing durometer of the tubular wall 502 between the distal segment and the proximal segment may create a sharp transition or a high stiff-to-flex ratio in the body 500. Thus, the body 500 can be relatively rigid at the proximal segment, but substantially pliable or flexible at the distal segment. The steerability of the body 500, the amount of force to bend the body 500, and/or the locality of the bend force and/or actuations may depend on the durometer of the body 500. A sharp transition may improve the steerability, the amount of force, and/or the locality of the force when the body 500 is in use. In some aspects, the tubular wall 502 may have a common or continuous durometer (e.g., between 30D and 80D) along its length. In some aspects, the tubular wall 502 may include multiple changes in durometer along its length.

The body 500 may further include a plurality of secondary lumens 506 extending longitudinally through a length of the tubular wall 502. The secondary lumens 506 may be shaped and sized to accommodate pullwires. Thus, the secondary lumens 506 may also be referred to as pullwire lumens. The secondary lumens 506 are positioned within the tubular wall 502 radially spaced apart by an angle of about 90 degrees. The arms 510 of the cross-shaped cross section of the primary lumen 508 may define the angular positions of the secondary lumens 506 within the body 500. For example, the secondary lumens 506 may be positioned between adjacent arms 510. The primary lumen 508 and the secondary lumens 506 can be lined with a lubricious lining material such as a polytetrafluoroethylene (PTFE) or other suitable material. The lining material may create surfaces with less friction for threading, delivery, and actuations of the pullwires or any other suitable diagnostic sensor assembly. In addition, the lining material can function as a support structure to prevent the primary lumen 508 and/or the secondary lumens 506 from collapsing. Further, the lining material can function as a barrier to protect abrasion caused by shifting or actuations of the pullwires and/or threading of the diagnostic sensor assembly.

The body 500 may further include a shield 504 embedded within the tubular wall 502. The shield 504 can be composed of any suitable material and geometry. In some aspects, the shield comprises at least one of a mu-metal, a metallic glass alloy, and/or other material having a magnetic permeability between 80,000 and 400,000 or greater. The shield 504 may comprise a cylindrical braid, a coil, a weave, and/or other suitable configuration. In this regard, the shield 504 may partially and/or fully surround wires 512 extending from the one or more electromagnetic position sensors along a length of the flexible elongate member. For example, as shown in FIG. 7, two pairs of wires 512 are shown. Each of the pairs of wires 512 is coupled to a corresponding electromagnetic position sensor. In some aspects, the wires 512 include an outer insulating layer. In some instances, the parameters (e.g., thickness, durometer, etc.) of the insulation layer and/or the twists per inch of the wires 512 may be selected based on desired flexibility and/or dielectric properties, which may be optimized for the specific applications and/or procedures, including limiting particular type(s) and/or frequencies of electromagnetic interference. The electromagnetic position sensors may have a known position relative to the imaging core, the distal portion of the flexible elongate member, another electromagnetic position sensor, the distal most tip, a boundary and/or middle of the tip assembly, a boundary and/or middle of the imaging core, a radiopaque marker, and/or other aspects or components of the intraluminal imaging device. In some aspects, the shield 504 may partially and/or fully surround a plurality of wires 514 extending from the imaging core to the proximal portion of the flexible elongate member. For example, as shown in FIG. 7, a bundle of wires 514 is shown extending from the imaging core to the proximal portion of the flexible elongate member. The bundle of wires 514 includes seventeen wires, but it is understood that the wires 514 may include any suitable number of wires, including between 1 and 65 or other suitable number. Further, the wires 514 may be part of or form a cable or otherwise be grouped or bundled together.

In some aspects, the shield 504 may have braids with pitches that vary along a length of the tubular wall 502. The braids can include any suitable braid pattern. The braid pattern may be selected to improve torque transmission, pushability, and/or kink resistance. For example, the braids at the distal portion may be configured to have a higher per inch count (PIC) than the braids at the proximal portion. The higher PIC at the distal portion may provide a greater flexibility, while the lower PIC at the proximal portion may provide a stiffer support structure. In some aspects, the distal portion has a first PIC, the proximal portion has a second PIC, and a transition portion between the proximal and distal portions has a varying PIC that smoothly transitions from the first PIC to the second PIC. The smooth transition in the braid pitches in the transition portion can alleviate a weak kink point that can result from an abrupt transition between a lower durometer, higher PIC distal segment and a higher durometer, lower PIC proximal segment.

In some aspects, the shield 504 is formed over an outer surface of the inner extrusion of the tubular wall 502. An outer extrusion of the tubular wall 502 may then be formed over the shield 504 to complete the formation of the tubular wall with the shield embedded therein. In this regard, the inner and outer extrusions may be formed of the same and/or different materials. In some aspects, the material used for the inner and/or outer extrusions may change along a length of the tubular wall 502 (e.g., higher durometer proximal segment, a lower durometer distal segment, and/or a transition segment between the proximal and distal segments). Similarly, the pitch and/or structure of the shield 504 may change along a length of the tubular wall 502 (e.g., higher PIC distal segment, lower PIC proximal segment, and/or transition segment between the proximal and distal segments).

Dimensions of the body 500 can vary in different aspects and/or based on intended uses and/or associated access approaches of the associated intraluminal imaging device. In some aspects, the body 500 may be a 9 Fr catheter. Thus, the body 500 can have an outer diameter of about 3 mm. In some aspects, the distal segment of the body 500 can have a length between about 70 mm to about 81 mm or any other suitable length, which may be based on a required or desired bend radius for the distal segment of the body 500. The proximal segment of the body 500 can have a length between about 872 mm to 877 mm or any other suitable length. The length of the body 500 and/or the relative lengths of the proximal and distal segments may be configured based on intended uses and/or associated access approaches of the associated intraluminal imaging device.

FIG. 8 is a cross-sectional end view of a body 600 of an intraluminal imaging device according to aspects of the present disclosure. In some aspects, the body 600 may be utilized in intracardiac echocardiography (ICE) catheters, intravascular ultrasound (IVUS) catheters, and/or other imaging devices having electromagnetic position tracking capabilities. For example, the body 600 may form part or all of flexible elongate member 108 of the ICE device 110, including the distal portion 104 and/or the proximal portion 106, or sections thereof. The body 600 includes many features the same as or similar to the body 500 described above with respect to FIGS. 5-7. For sake of brevity, the description of common or similar features will not be repeated, though the same reference numerals will be utilized in some instances.

The body 600 may include a reinforcement element 604 in the tubular wall 502 and a shield 606 within the primary lumen 508. The reinforcement element 604 may be formed of stainless steel, aluminum, copper, or other material having a magnetic permeability lower than the shield 606. For example, in some instances the reinforcement element 604 may have a magnetic permeability of approximately 1.0. The reinforcement element 604 may provide desired structural support to the tubular wall 502 and/or the body 600. In some instances, the reinforcement element 604 may have braids with pitches that vary along a length of the tubular wall 502. The braids can include any suitable braid pattern. The braid pattern may be selected to improve torque transmission, pushability, and/or kink resistance. For example, the braids at the distal portion may be configured to have a higher per inch count (PIC) than the braids at the proximal portion. The higher PIC at the distal portion may provide a greater flexibility, while the lower PIC at the proximal portion may provide a stiffer support structure. In some aspects, the distal portion has a first PIC, the proximal portion has a second PIC, and a transition portion between the proximal and distal portions has a varying PIC that smoothly transitions from the first PIC to the second PIC. The smooth transition in the braid pitches in the transition portion can alleviate a weak kink point that can result from an abrupt transition between a lower durometer, higher PIC distal segment and a higher durometer, lower PIC proximal segment.

As shown, the shield 606 is positioned within the primary lumen 508. The shield 606 surrounds the pairs of wires 512 extending from the electromagnetic position sensors. The shield 606 also surrounds the plurality of wires 514 extending from the imaging core. In some aspects, the shield 606 comprises at least one of a mu-metal, a metallic glass alloy, and/or other material having a magnetic permeability between 80,000 and 400,000 or greater. The shield 606 may comprise a cylindrical braid, a coil, a weave, and/or other suitable configuration. In this regard, the shield 606 may partially and/or fully surround the pairs of wires 512 and/or the plurality of wires 514. In some aspects, the shield 606 may be embedded within a tubular member sized and shaped for positioning within the primary lumen 508. In some aspects, the shield 606 may be used in combination with the shield 504 of the body 500 described above. That is, instead of reinforcement element 604, the body 600 may include the shield 504 in some instances.

FIG. 9 is a cross-sectional end view of a body 700 of an intraluminal imaging device according to aspects of the present disclosure. In some aspects, the body 700 may be utilized in intracardiac echocardiography (ICE) catheters, intravascular ultrasound (IVUS) catheters, and/or other imaging devices having electromagnetic position tracking capabilities. For example, the body 700 may form part or all of flexible elongate member 108 of the ICE device 110, including the distal portion 104 and/or the proximal portion 106, or sections thereof. The body 700 includes many features the same as or similar to the body 500 and the body 600 described above with respect to FIGS. 5-8. For sake of brevity, the description of common or similar features will not be repeated, though the same reference numerals will be utilized in some instances.

The body 700 may include a reinforcement element 604 in the tubular wall 502 and a shield 706 within the primary lumen 508. As shown, the shield 706 is positioned within the primary lumen 508. The shield 706 surrounds the pairs of wires 512 extending from the electromagnetic position sensors. The shield 706 does not surround the plurality of wires 514 extending from the imaging core and, therefore, separates the wires 512 from the plurality of wires 514. In this manner, the shield 706 may serve to reduce and/or eliminate potential electromagnetic interference in the wires 512 caused by the plurality of wires 514. In some aspects, the shield 706 comprises at least one of a mu-metal, a metallic glass alloy, and/or other material having a magnetic permeability between 80,000 and 400,000 or greater. The shield 706 may comprise a cylindrical braid, a coil, a weave, and/or other suitable configuration. In this regard, the shield 706 may partially and/or fully surround the pairs of wires 512. In some aspects, the shield 706 may be embedded within a tubular member sized and shaped for positioning within the primary lumen 508. In some aspects, the shield 706 may be used in combination with the shield 504 of the body 500 and/or the shield 606 of the body 600 described above.

FIG. 10 is a schematic diagram of an intraluminal imaging system 800 according to aspects of the present disclosure. The intraluminal imaging system 800 includes an intraluminal imaging device (e.g., intracardiac catheter, intracardiac echocardiography (ICE) catheter, intravascular ultrasound (IVUS) catheter, and/or other imaging device) having electromagnetic position tracking capabilities. For example, the intraluminal imaging system 800 may include an intraluminal imaging device including one or more features discussed above in FIGS. 1-9. In this regard, an intracardiac catheter may be sized and shaped for advancement through a blood vessel and include an imaging core and one or more electromagnetic position sensors coupled to a distal portion of the intracardiac catheter. The intraluminal imaging system 800 also includes a multiplexer 802, a demultiplexer 804, and a magnetic positioning visualization system 806. The magnetic positioning visualization system 806 may include the electromagnetic field generator 420, the processing system 130, and/or the monitor 132 described above. In some aspects, the magnetic positioning visualization system 806 may include a processing system and/or a display separate from the processing system 130 and/or the monitor 132.

The multiplexer 802 may be positioned within a proximal portion and/or handle of the intraluminal imaging device. The demultiplexer 804 may be positioned within a hardware component (e.g., the processing system 130 and/or the magnetic positioning visualization system 806) in communication with the multiplexer 802. In some instances, the multiplexer 802 and the demultiplexer 804 are connected via one or more wires 808. The wires 808 may be part of a cable and/or other component extending between the multiplexer 802 and the demultiplexer 804 external to the intraluminal imaging device. As shown, the multiplexer 802 may be in communication with one or more electromagnetic position sensors 140 via twisted pairs of wires 512. In this regard, the multiplexer 802 may be configured to perform time-division multiplexing to remove electromagnetic interference within the wires 512 induced along the length of the intraluminal imaging device (as indicated by reference numeral 810). In some aspects, the multiplexer 802 is configured to perform time-division multiplexing with a time slot length between about 0.25 microseconds and about 1.0 microsecond. The multiplexer 802 may be configured to perform the time-division multiplexing by subtracting a signal based on an immediately preceding time slot from a signal based on a current time slot. The multiplexer 802 may be further configured to receive and process analog input signals from the wires 512 and output analog output signals to the demultiplexer 804 via the wires 808.

The demultiplexer 804 may be configured to demultiplex the analog output signals received from multiplexer 802. Further, the demultiplexer 804 and/or a processing unit in communication with the demultiplexer 804 may be configured to remove electromagnetic interference within the wires 808 induced along the length of the cable or other device external to the intraluminal imaging device (as indicated by reference numeral 812). For example, in some instances, the electromagnetic interference within the wires 808 may be reduced and/or eliminated by subtracting time-shifted signals to reduce common noise. By reducing the electromagnetic interference within the intraluminal imaging device and/or external to the intraluminal imaging device, the spatial accuracy of the determined positions and/or orientations of the electromagnetic position sensors 140 may be improved. This can lead to better diagnostic results that, in turn, lead to better treatment plans and associated patient outcomes. Further, the improved spatial accuracy can reduce procedure times and improve the operator's experience with using the intraluminal imaging system 800.

Persons skilled in the art will recognize that the apparatus, systems, and methods described above can be modified in various ways. Accordingly, persons of ordinary skill in the art will appreciate that the aspects encompassed by the present disclosure are not limited to the particular exemplary aspects described above. In that regard, although illustrative aspects have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraluminal imaging device, comprising:
a flexible elongate member;
an imaging core coupled to a distal portion of the flexible elongate member;
one or more electromagnetic position sensors coupled to the distal portion of the flexible elongate member, the one or more electromagnetic position sensors having a known position relative to at least one of the imaging core, the distal portion of the flexible elongate member, or another electromagnetic position sensor of the one or more electromagnetic position sensors;
a handle coupled to a proximal portion of the flexible elongate member;
a pair of wires extending from each of the one or more electromagnetic position sensors to a proximal portion of the flexible elongate member; and
a shield extending along a length of the flexible elongate member from the distal portion to the proximal portion, the shield having a magnetic permeability between about 80,000 and about 400,000.

2. The intraluminal imaging device of claim 1, wherein the shield comprises a cylindrical braid.

3. The intraluminal imaging device of claim 1, wherein the shield comprises a coil.

4. The intraluminal imaging device of claim 1, wherein the shield comprises at least one of a mu-metal or a metallic glass alloy.

5. The intraluminal imaging device of claim 1, wherein the shield surrounds the pair of wires extending from each of the one or more electromagnetic position sensors to the proximal portion of the flexible elongate member.

6. The intraluminal imaging device of claim 5, wherein the shield is embedded within a tubular wall of the flexible elongate member.

7. The intraluminal imaging device of claim 6, wherein the tubular wall defines an outer surface of the flexible elongate member.

8. The intraluminal imaging device of claim 5, wherein the shield surrounds a plurality of wires extending from the imaging core to the proximal portion of the flexible elongate member.

9. The intraluminal imaging device of claim 5, wherein a plurality of wires extending from the imaging core to the proximal portion of the flexible elongate member are positioned outside of the shield.

10. The intraluminal imaging device of claim 1, further comprising:
a multiplexer positioned within the handle or the proximal portion of the flexible elongate member, the multiplexer configured to process signals carried by the pair of wires extending from each of the one or more electromagnetic position sensors to remove electromagnetically induced noise.

11. An intraluminal imaging device, comprising:
a flexible elongate member;
an imaging core coupled to a distal portion of the flexible elongate member;
one or more electromagnetic position sensors coupled to the distal portion of the flexible elongate member, the one or more electromagnetic position sensors having a known position relative to at least one of the imaging core, the distal portion of the flexible elongate member, or another electromagnetic position sensor of the one or more electromagnetic position sensors;
a handle coupled to a proximal portion of the flexible elongate member;
a pair of wires extending from each of the one or more electromagnetic position sensors to a proximal portion of the flexible elongate member; and
a multiplexer positioned within the handle or the proximal portion of the flexible elongate member, the multiplexer configured to process signals carried by the pair of wires extending from each of the one or more electromagnetic position sensors to remove electromagnetically

12. The intraluminal imaging device of claim 11, wherein the multiplexer is configured to perform time-division multiplexing with a time slot length between about 0.25 microseconds and about 1.0 microsecond.

13. The intraluminal imaging device of claim 12, wherein the multiplexer is further configured to perform the time-division multiplexing by subtracting a signal based on an immediately preceding time slot from a signal based on a current time slot.

14. The intraluminal imaging device of claim 13, wherein the multiplexer is further configured to receive analog input signals and output analog output signals, wherein the analog input signals include the signal based on the immediately preceding time slot and the signal based on the current time slot.

15. An apparatus, comprising:
an intracardiac catheter sized and shaped for advancement through a blood vessel;
an imaging core coupled to a distal portion of the intracardiac catheter;
one or more electromagnetic position sensors coupled to the distal portion of the intracardiac catheter, the one or more electromagnetic position sensors having a known position relative to at least one of the imaging core, the distal portion of the intracardiac catheter, or another electromagnetic position sensor of the one or more electromagnetic position sensors;
a handle coupled to a proximal portion of the intracardiac catheter;
a pair of wires extending from each of the one or more electromagnetic position sensors to a proximal portion of the intracardiac catheter;
a shield extending along a length of the intracardiac catheter from the distal portion to the proximal portion, the shield having a magnetic permeability between about 80,000 and about 400,000; and
a multiplexer positioned within the handle or the proximal portion of the intracardiac catheter, the multiplexer configured to process signals carried by the pair of wires extending from each of the one or more electromagnetic position sensors to remove electromagnetically
